(19) United States
(12) Patent Application Publication
(10) Pub. No.: US 2003/0181809 A1
(43) Pub. Date: Sep. 25, 2003

(54) 3D imaging for catheter interventions by use of 2D/3D image fusion

(76) Inventors: Andrew F. Hall, St. Charles, MO (US); John Rauch, St. Louis, MO (US); Joachim Hornegger, Baiersdorf (DE); Reinmar Killmann, Forchheim (DE); Norbert Rahn, Forchheim (DE); Johann Seissl, Erlanger (DE); Siegfried Wach, Hochstadt (DE); Benno Heigl, Untersiemau (DE)

Correspondence Address: Harness, Dickey & Pierce, P.L.C., 7700 Bonhomme, Suite 400, St. Louis, MO, 63105, US

(21) Appl. No.: 10/290,112

(22) Filed: Nov. 7, 2002

(30) Foreign Application Priority Data
Mar. 11, 2002 (DE) 10210646.0

(51) Int. Cl. A61B 5/05

(52) U.S. Cl. 600/425

(57) ABSTRACT

A method of visualizing a medical instrument that has been introduced into an area of examination within a patient

The subject matter of the present invention relates to a method of visualizing a medical instrument that has been introduced into an area of examination within a patient, in particular a catheter that is used during a cardiological examination or treatment, comprising the following steps:

using a 3D image data set of the area of examination and generating a 3D reconstructed image of the area of examination,

taking at least one 2D X-ray image of the area of examination in which the instrument is visualized,

registering the 3D reconstructed image relative to the 2D X-ray image, and

visualizing the 3D reconstructed image and superimposing the 2D X-ray image over the 3D reconstructed image on a monitor.

The subject matter of the present invention relates to a method of visualizing a medical instrument that has been introduced into an area of examination within a patient, in particular a catheter that is used during a cardiological examination or treatment.

Patients suffering from disorders are increasingly examined or treated by means of minimally invasive methods, i.e., methods that require the least possible surgical intervention. One example is treatment with endoscopes, laparoscopes, or catheters which are introduced into the area of examination inside the patient via a small opening in the body. Catheters are frequently used in cardiological examinations, for example, in the presence of cardiac arrhythmias which are today treated by means of so-called ablation procedures.

In such procedures, a catheter is introduced into a chamber of the heart under radiological guidance, i.e., by taking X-ray images via veins or arteries. In the cardiac chamber, the tissue that causes the arrhythmia is ablated by means of application of high-frequency electric current, which leaves the previously arrhythmogenic substrate behind in the form of necrotic tissue. The healing character of this method has significant advantages when compared to lifelong medication; in addition, this method is also economic in the long term.

From the medical and technical standpoint, the problem is that although during the intervention the catheter can be visualized very accurately and with high resolution in one or several X-ray images, which are also called fluoro images, the anatomy of the patient can only be inadequately visualized on the X-ray images. To track the catheter, generally two 2D X-ray images from two different directions of projection, in most cases orthogonal to each other, have so far been taken. Based on the information provided by these two images, the physician himself now has to determine the position of the catheter, something that is often accompanied by considerable uncertainty.

The problem to be solved by the present invention is to make available a possible visualization technique which makes it easier for the physician to observe the exact position of the instrument, i.e., of the catheter in the heart, in the area of examination.

To solve this problem, a method of the type mentioned in the introduction using the following steps is made available:

using a 3D image data set of the area of examination and generating a 3D reconstructed image of the area of examination,

taking at least one 2D X-ray image of the area of examination in which the instrument is visualized,

registering the 3D reconstructed image relative to the 2D X-ray image, and

visualizing the 3D reconstructed image and superimposing the 2D X-ray image over the 3D reconstructed image on a monitor.

The method according to the present invention makes it possible during the examination to visualize the instrument, i.e., the catheter (hereinafter, reference will be exclusively made to a catheter), practically in real-time in the correct position on a three-dimensional image of the area of examination, for example, the heart or the central vascular tree of the heart, etc. This is made possible by the fact that a three-dimensional reconstructed image of the area of examination is generated using a 3D image data set, on the one hand, and that the 2D X-ray image which is taken during the intervention is superimposed over this 3D reconstructed image. Since both images are correctly registered, which means that the coordinate systems of these images are correlated with respect to each other, the superimposition with the simultaneous insertion of the catheter in the accurate position into the 3D image is possible. As a result, the physician can very accurately visualize the catheter in its actual position in the area of examination, the relevant anatomical details of which he can also see very accurately and in high resolution. This makes possible an easy navigation of the catheter, specific areas, e.g., sites in which an ablation needs to be carried out, can be accurately targeted, etc.

According to the present invention, the 3D image data set may be a data set that was acquired prior to the operation. This means that the data set may have been acquired at any time prior to the actual intervention. Any 3D image data set, regardless of the acquisition modality, i.e., a CT, MR, or 3D angiographic X-ray image data set, can be used. All of these data sets allow an exact reconstruction of the area of examination, thus making it possible to visualize this area with anatomic accuracy. As an alternative, it is also possible to use an intraoperatively acquired image data set in the form of a 3D angiographic X-ray image data set. In this context, the term “intraoperative” indicates that this data set is acquired during the same time in which the actual intervention is carried out, i.e., when the patient is already lying on the operating table but before the catheter is inserted, which, however, will take place very shortly after the 3D image data set has been acquired.

If the area of examination is an area which moves rhythmically or arrhythmically, for example, the heart, care must be taken to ensure that in order to visualize the area of examination accurately, the 3D reconstructed image and the 2D X-ray image or images that is or are to be taken or superimposed show the area of examination in the same phase of motion or were taken in the same phase of motion. For this purpose, provision can be made to acquire the phase of motion in addition to the 2D X-ray image and, for the reconstruction of the 3D reconstructed image, to use only those image data which had been taken in the same phase of motion as the 2D X-ray image. This means that in order to obtain or superimpose images or volumes in correct phase relation to one another, the phase of motion must be acquired both when the 3D image data set is taken and when the 2D X-ray image is taken. The reconstruction and the image data used for this purpose are dependent on the phase in which the 2D X-ray image was taken. One example of an acquisition of the phase of motion is an ECG which is taken parallel [to the X-ray image] and which records the movements of the heart. Based on the ECG, it is subsequently possible to select the relevant image data. To take the 2D X-ray images, the image-taking device can be triggered via the ECG, which ensures that consecutively taken 2D X-ray images are always taken in the same phase of motion. Alternatively, it is also possible to record the respiratory phases of the patient as the phase of motion. This can be accomplished, for example, using a respiration belt which is worn around the chest of the patient and which measures the movement of the thorax; as an alternative, it is also possible to use position sensors on the chest of the patient in order to record said phase of motion.

Furthermore, it is useful if, in addition to the phase of motion, the time at which the 2D X-ray image is taken is recorded and if only those image data that were taken at the same time as the 2D X-ray image are used to reconstruct the 3D reconstructed image. The heart, when it contracts, changes its shape within one phase of motion of, for example, one second only within a relatively narrow time window; the rest of the time, the heart retains its shape. Thus, using the time as an additional dimension, is not now possible to obtain a nearly film-like threedimensional visualization of the heart, since the corresponding 3D reconstructed image can be reconstructed at any time and a relevant 2D X-ray image that had been taken at the same time can be superimposed. In the final analysis, one thus obtains a nearly film-like visualization of the beating heart, superimposed by a film-like visualization of the guided catheter. This means that at different times within one phase of motion of the heart, a separate phase- and time-specific 3D reconstructed image is generated; in addition, several phase- and time-specific 2D X-ray images are taken, with a 2D X-ray image being superimposed over a 3D image that was reconstructed in the same phase and at the same time so that the instrument in the moving heart is visualized by consecutively displaying the 3D reconstructed images and superimposing the 2D X-ray images.

To register the two images, various approaches are feasible. First of all, it is possible to identify at least one anatomic image element or several markings in the 2D X-ray image and to identify the same anatomic image element or the same markings in the 3D reconstructed image and to orient the 3D reconstructed image relative to the 2D X-ray image by means of translation and/or rotation and/or 2D projection. It is possible to use, e.g., the surface of the heart as the anatomic image element, which means that a so-called “figure-based” registration takes place in that after identification of the anatomic image element, the 3D reconstructed image is rotated and translated and possibly changed in its projection until its position corresponds to that of the 2D X-ray image. Markings to be used include so-called landmarks, and said landmarks can be anatomic markings. Examples include specific vascular branching points or small segments of coronary arteries and similar markings which can be interactively defined by the physician in the 2D X-ray image and which are subsequently searched for and identified in the 3D reconstructed image by means of suitable analytical algorithms, after which the orientation takes place. Landmarks that are not anatomical landmarks include, e.g., any other markings as long as they are recognizable both in the 2D X-ray image and in the 3D reconstructed image. Depending on whether or not the intrinsic parameters of the device that takes the 2D X-ray images are known, it suffices to identify at least four landmarks if these parameters (distance from focus to detector, pixel size of a detector element, point of penetration of the center beam of the X-ray tube on the detector) are known. If these parameters are not known, a minimum of six markings in each picture must be identified.

Another possibility of registering the images provides for the use of two 2D x-ray images which are positioned at a certain angle, preferably 90°, relative to each other and in which several identical markings are identified, the 3D volume position of which is determined by means of back projection, after which the 3D reconstructed image in which the same markings are identified are oriented by means of translation and/or rotation and/or 2D projection relative to the 3D positions of the markings. In this case, in contrast to the 2D/3D registration described earlier, a 3D/3D registration is carried out on the basis of the volume positions of the markings. The volume positions follow from the points of intersection of the straight lines generated by the back projection which run from the relevant marking identified in the 2D X-ray image to the tube focus.

Another possibility is the so-called “image-based” registration. In this case, the 3D reconstructed image is used to generate a 2D projection image in the form of a digitally reconstructed radiogram (DRR) which is compared to the 2D X-ray image for similarities, and for the purpose of optimizing the registration, the similarity between the 2D projection image and the 2D X-ray image is moved by means of translation and/or rotation until the similarities reach a predetermined minimum level of similarity. It is useful if after its generation, the 2D projection image—by means of user guidance—is moved into a position in which it most closely resembles the 2D X-ray image, and if subsequently the optimization cycle is initiated in order to shorten the computing time needed for the registration. Instead of user-guided rough positioning, it is also possible to record the position-specific parameters used to take the 2D X-ray image, e.g., the position of the C-shaped arm and its orientation via suitable means of taking the image. Depending on this information, a rough position can subsequently be determined by the computer. Every time the degree of similarity is calculated and it is found that the predetermined minimum level of similarity is not yet reached, the parameters of the transformation matrix for the transformation of the 2D projection image to the 2D X-ray image are newly calculated and modified in order to increase the level of similarity. The similarity can be determined, for example, on the basis of the local distribution of gray-scale intensity values. But any other method of determining the degree of similarity that can be implemented via suitable computer algorithms can be used.

To generate the 3D reconstructed image which is the basis for the subsequent superimposition, different possibilities are available. According to one approach, this image is generated in the form of a perspective maximum-intensity projection image. Alternatively, it is generated in the form of a perspective volume-rendering projection image (VRT). In both cases, it is possible for the user to select from the 3D reconstructed image of any type an area over which the 2D X-ray image is superimposed. This means that the physician is able to choose on the 3D reconstructed image any area over which the 2D X-ray image is subsequently superimposed. In the case of a MIP image, this means that during the visualization, the thickness can be interactively changed; in the case of a VRT image, interactive clipping can be done during the visualization.

Another possibility is to select from the 3D reconstructed image a specific layer plane image over which the 2D X-ray image is superimposed. In this case, the physician can choose a layer image with a certain thickness from any area of the image and have it displayed for superimposition.

According to another approach, the user can choose from several phase- and time-specific 3D reconstructed images (i.e., images which show the heart or a similar organ in different phases and at different times) a specific layer plane image, with the layer plane images being displayed one after the other and with the associated phase- and time-specific 2D X-ray images being superimposed. Here, the different 3D reconstructed images always display the same layer plane, but at different times and thus in different cardiac phases, and these images can be superimposed on the associated 2D X-ray image. An alternative approach provides that the user can select from the 3D reconstructed image several consecutive layer plane images which, when assembled, show part of the heart; these images can subsequently be superimposed one after the other over a 2D X-ray image. In this case, only one reconstructed [sic] 3D reconstructed image which was taken in a specific phase at a specific time is used, but a stack of layers which can be interactively chosen by the user is selected from it. This stack of layers is now superimposed one image after the other over an associated 2D X-ray image which corresponds in phase and time at which it was taken to the reconstructed image. Thus, the physician so-to-speak is faced with a stepwise display, with which he moves through the area of examination taken, in a way as though he were viewing a film.

Since the catheter or, quite generally, the instrument is the important information element in the 2D X-ray image, it is useful to highlight said catheter or instrument prior to superimposition in the X-ray image by increasing the contrast so that it is clearly visibly in the superimposed image. It is especially useful if the instrument is automatically segmented from the 2D X-ray image by means of image analysis so that only the instrument is superimposed over the 3D reconstructed image. This is beneficial in that the high-resolution 3D reconstructed image is in no way affected by the superimposition. It is, by the way, also possible for the instrument to be displayed in color or to blink in the superimposed image so as to make it even more recognizable.

Based on the possibility of visualizing the instrument in the correct position in the area of examination, it is also possible to use this method to document the treatment in a reproducible manner. If, for example, the instrument used is an ablation catheter, a 2D X-ray image of the ablation catheter located at an ablation area can be stored together with a 3D reconstructed image, possibly in the form of a superimposed image. Thus, later on, it will be clearly visible where the ablation area was located. If an ablation catheter is used with an integrated device for recording an intracardial ECG, it is also possible to store the ECG data which were recorded in the ablation areas together with the superimposed image. The intracardial ECG data differ in different positions of the heart, thus again making it possible to identify each position relatively accurately.

In addition to the method according to the present invention, this invention also makes available a medical examination and/or treatment device which is designed to carry out the method.

Other advantages, features, and details of this invention follow from the practical examples described below as well as from the drawings. As can be seen:

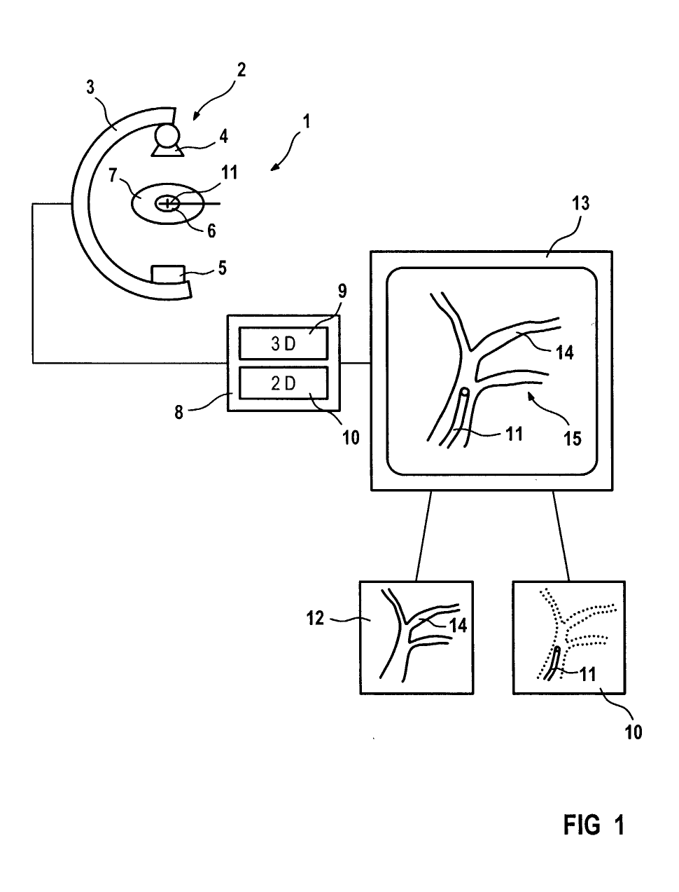

FIG. 1 shows a schematic sketch of a medical examination and/or treatment device according to the present invention,

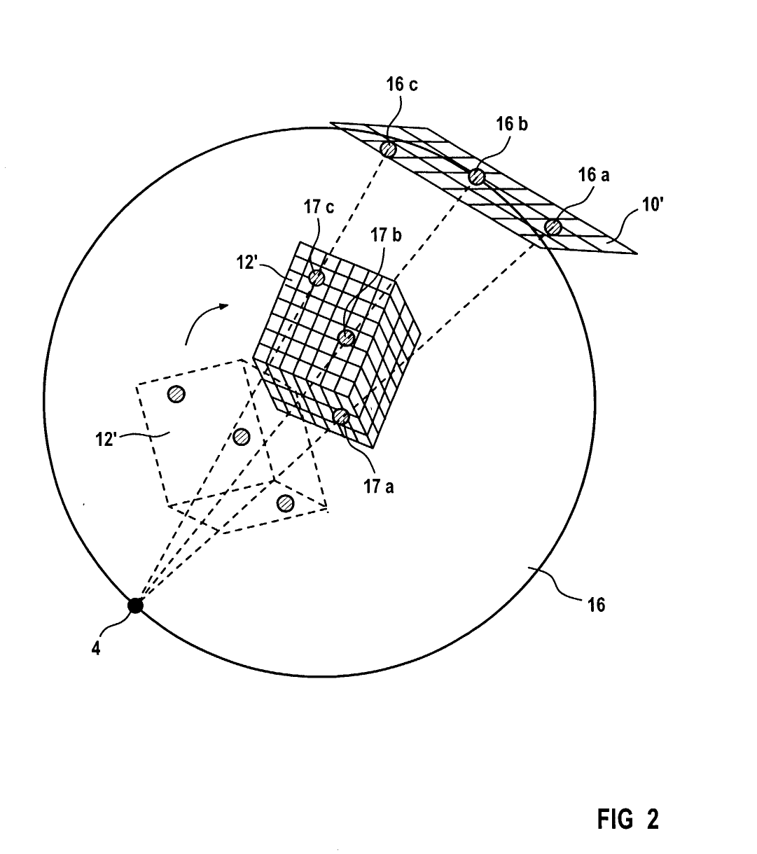

FIG. 2 shows a schematic sketch which explains the registration of the 3D reconstructed image relative to a 2D X-ray image, and

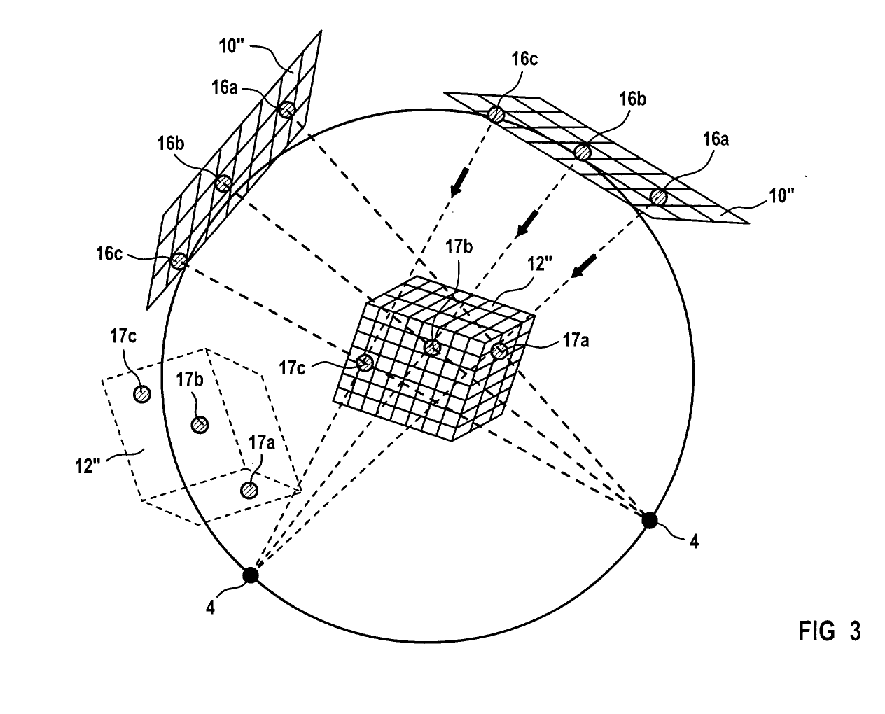

FIG. 3 shows a schematic sketch which explains the registration of the 3D reconstructed image relative to two 2D X-ray images.

FIG. 1 is schematic sketch of an examination and/or treatment device 1 according to the present invention, in which only the essential components are shown. The device comprises an image-taking device 2 for taking two-dimensional X-ray images. It has a C-shaped arm 3, to which an X-ray radiation source 4 and a radiation detector 5, e.g., a solid state image detector, are attached. The area of examination 6 of patient 7 is located essentially in the isocenter of the C-shaped arm so that it is fully visible in the 2D X-ray image.

The operation of device 1 is controlled by a control and processing device 8 which, among other things, also controls the image-taking operation. It also comprises an image processing device which is not shown in the drawing. In this image processing device, a 3D image data set 9 which was preferably acquired prior to the intervention is available. This image data set may have been acquired by means of any examination modality, for example, a computer tomography scanner or an NMR tomograph or a 3D angiographic device. The data set may also be taken as a so-called intraoperative data set, using the image-taking device 2 [of the examination and treatment device according to the present invention], i.e., immediately prior to the actual catheter intervention, in which case the image-taking device 2 is operated in the 3D angiography mode.

In the example shown, a catheter 11 is introduced into the area of examination 6, which in this case is the heart. This catheter is visible in the 2D X-ray image 10 which in FIG. 1 is magnified and shown in the form of a schematic sketch.

What is not seen in the 2D X-ray image 10, however, is the anatomic structure surrounding catheter 11. To also visualize this anatomic structure, a 3D reconstructed image 12 which is also magnified in the schematic sketch of FIG. 1, is generated from 3D image data set 9 using known methods of reconstruction. This reconstructed image can be generated, for example, as an MIP image or as a VRT image.

On a monitor 13, the 3D reconstructed image 12 in which the surrounding anatomic structure—here a vascular tree 14 of the heart—can be seen as a three-dimensional image. Over this image, the 2D X-ray image 10 is superimposed. Both images are registered relative to each other. I.e., in superimposition image 15, catheter 11 is shown in the exact correct position and orientation with respect to vascular tree 14. Thus, the physician can see exactly where the catheter is located and how he may have to continue navigating it or how and where the treatment is to be started or continued.

Catheter 11 can be shown in any emphasized form to ensure that it is unambiguously and well recognizable. Thus, it may be emphasized by contrast, or it may be displayed in color. Also, using suitable object or boundary detection algorithms as part of an image analysis, it may be possible not to superimpose the entire X-ray image 10 [over the other image] but to segment catheter 11 from X-ray image 10 and to superimpose only this catheter over the 3D reconstructed image.

FIG. 2 shows one possibility by which the 3D reconstructed image and the 2D X-ray image can be registered. What is shown is a 2D reconstructed image 10′ which was taken in the same position by detector 5 (not shown). Also shown is X-ray radiation source 4 and its focus and motion path 16 around which the detector and the source are moved by means of C-shaped arm 3.

Also shown is the reconstructed 3D reconstructed image 12′ immediately before it was generated, without it having been registered relative to the 2D X-ray image 10′.

To register the image, several—in the example shown, three markings or landmarks 16a, 16b, and 16c—are identified or defined in the 2D X-ray image 10′. As landmarks, it is possible to use, e.g., anatomic markings, such as certain vascular branching points, etc. These landmarks are now also identified in the 3D reconstructed image 12′. As can be seen, landmarks 17a, b, c are located in positions in which they do not coincide directly with the projection beams which run from radiation source 4 to landmarks 16a, b, c in the 2D X-ray image 10′. If landmarks 17a, b, c were to be projected onto the detector plane, they would be seen in positions that clearly differ from landmarks 16a, b, c.

To register the image by means of the rigid registration technique, 3D reconstructed image 12′ is moved by means of translation and rotation until landmarks 17a, b, c can be projected onto landmarks 16a, b, c. Thereafter, the registration is concluded. The orientation of the registered 3D reconstructed image 12′ is shown by means of the exploded representation of the reconstructed image which in this figure is only diagrammatically shown in the form of a cube.

FIG. 3 shows another possibility of image registration. In this case, two 2D X-ray images 10″ are used which had been taken in two different X-ray radiation source-detector positions. They are preferably orthogonal to each other. The positions of X-ray radiation source 4 are shown, and from these positions, the positions of the radiation detector follow.

In each 2D X-ray image, the same landmarks 16a, 16b, 16c are identified. Corresponding landmarks 17a, 17b, 17c are also identified in the 3D reconstructed image 12″. Next, for image registration, the 3D volume positions of landmarks 16a, 16b, 16c are identified. In the ideal case, these are found in the points of intersection of the projection beams of each respective landmark 16a, 16b, 16c and the focus of X-ray radiation source 4. Shown are the volume positions of landmarks 16a, 16b, 16c which are located around the isocenter of the C-shaped arm.

If the lines do not intersect exactly, the associated volume positions can be defined by means of suitable approximation techniques. For example, it is possible to define a volume position as the location in which the distance between the two lines which ideally intersect is smallest, or by a similar technique.

For image registration, the 3D reconstructed image 12″ is again moved by means of rotation and translation and possibly by means of 2D projection (i.e., scaling according to size) until landmarks 17a, 17b, 17c and the volume positions of landmarks 16a, 16b, 16c are congruent. Again, in this figure, this is shown by means of the exploded representation of the 3D reconstructed image 12″.

Once the registration—no matter which method was used—is concluded, the positions can be correctly superimposed over each other, as described in the context of FIG. 1.

1. A method of visualizing a medical instrument that has been introduced into an area of examination within a patient, in particular a catheter that is used during a cardiological examination or treatment, comprising the following steps:
using a 3D image data set of the area of examination and generating a 3D reconstructed image of the area of examination,
taking at least one 2D X-ray image of the area of examination in which the instrument is visible,
registering the 3D reconstructed image relative to the 2D X-ray image, and
visualizing the 3D reconstructed image and superimposing the 2D X-ray image over the 3D reconstructed image on a monitor.

2. The method as claimed in claim 1 in which the 3D image data set used is a preoperatively acquired data set or an intraoperatively acquired data set.

3. The method as claimed in claim 1 or 2 in which, in an area of examination which moves rhythmically or arrhythmically, the phase of motion, in addition to the 2D X-ray image, is recorded and only those image data, which were recorded in the same phase of motion as the 2D X-ray image, are used to reconstruct the 3D reconstructed image.

4. The method as claimed in claim 3 in which, in addition to the phase of motion, the time at which the 2D X-ray image was taken is recorded and only those image data, which were recorded at the same time as the 2D X-ray image, are used to reconstruct the 3D reconstructed image.

5. The method as claimed in claim 3 or 4 where the area of examination is the heart and where, to record the phase of motion and potentially the time, an ECG is taken, as a function of which the taking of the 2D X-ray image is triggered, and where, to generate the 3D reconstructed image, an ECG is also dedicated to the image data while these are being acquired.

6. The method as claimed in claim 4 where the area of examination is the heart and a separate phase- and time-specific 3D reconstructed image is generated at different times within one cycle of motion, and where several phase- and time-specific 2D X-ray images are taken, with a 3D reconstructed image which was taken in the same phase and at the same time being superimposed over a 2D X-ray image so that by displaying the 3D reconstructed images one after the other and by superimposing the 2D X-ray images, the instrument in the moving heart is visualized.

7. The method as claimed in any one of the preceding claims in which, to register the 2D X-ray image, at least one anatomic image element or several markings is or are identified and the same anatomic image element or the same markings is or are identified, after which the 3D reconstructed image is oriented with respect to the 2D X-ray image by means of translation and/or rotation and/or 2D projection.

8. The method as claimed in any one of claims 1 through 6 in which for registration, two 2D X-ray images which are positioned at a certain angle, preferably at 90°, to each other are used in which two images several identical markings are identified, the 3D volume position of which is determined by back projection, after which the 3D reconstructed image, in which the same markings are identified, are oriented with respect to the 3D positions of the markings by means of translation and/or rotation and/or 2D projection.

9. The method as claimed in any one of claims 1 through 6 in which, to register the 3D reconstructed image, a 2D projection image in the form of a digital reconstructed radiograms is generated, which digital reconstructed radiogram is compared to the 2D X-ray image for similarities, whereby, to optimize the degree of similarity, the 2D projection image is moved by means of translation and/or rotation relative to the 2D X-ray image until the similarities reach a predetermined minimum level.

10. The method as claimed in claim 9 in which, by means of user guidance, the 2D projection image, after its generation, is first moved into a position in which it resembles the 2D X-ray image as much as possible, after which the optimization cycle is initiated.

11. The method as claimed in any one of the preceding claims in which the 3D reconstructed image is generated in the form of a perspective maximum intensity projection image.

12. The method as claimed in any one of claims 1 through 10 in which the 3D reconstructed image is generated in the form of a perspective volume-rendering projection image.

13. The method as claimed in claim 11 or 12 in which the user chooses from the 3D reconstructed image an area over which the 2D X-ray image is superimposed.

14. The method as claimed in claim 11 or 12 in which the user can choose from the 3D reconstructed image a specific layer plane image over which the 2D X-ray image is superimposed.

15. The method as claimed in claim 11 or 12 in which the user can choose from several phase- and time-specific 3D reconstructed images specific layer plane images which are displayed one after the other and over which the associated phase- and time-specific 2D X-ray images are superimposed.

16. The method as claimed in claim 11 or 12 in which the user can choose from a 3D reconstructed image several consecutive layer plane images which, when assembled, display a portion of the heart and which are one after the other superimposed over a 2D X-ray image.

17. The method as claimed in any one of the preceding claims in which the instrument, prior to superimposition, is emphasized in the 2D X-ray image by means of increased contrast.

18. The method as claimed in any one of the preceding claims in which the instrument, by means of image analysis, is segmented from the 2D X-ray image and only the instrument is superimposed over the 3D reconstructed image.

19. The method as claimed in any one of the preceding claims in which the instrument in the superimposition image blinks or is displayed in color.

20. The method as claimed in any one of the preceding claims in which the instrument used is an ablation catheter, whereby a 2D X-ray image with the ablation catheter located in an ablation area is stored together with a 3D reconstructed image.

21. The method as claimed in any one of the preceding claims in which the instrument used is an ablation catheter with an integrated device for taking an ECG during the intervention, whereby at least the ECG data that were recorded in the ablation areas are stored together with the superimposition image.

22. A medical examination and/or treatment device which is designed to carry out the method as claimed in any one of claims 1 through 21.
